(19) United States
(12) Patent Application Publication
(10) Pub. No.: US 2009/0011053 A1
(43) Pub. Date: Jan. 8, 2009

(54) METHOD OF DELETING nef GENE IN HIV-1 USING RED GINSENG

(76) Inventors: Young-Keol Cho, Seoul (KR); Heungsup Sung, Seoul (KR); You-Sun Jung, Seoul (KR)

Correspondence Address: THELEN LLP, P.O. BOX 640640, SAN JOSE, CA, 95164-0640, US

(21) Appl. No.: 11/773,967

(22) Filed: Jul. 6, 2007

(51) Int. Cl. A61K 36/258; A61P 31/18

(52) U.S. Cl. 424/728

(57) ABSTRACT

A method for inducing defective nef genes in HIV-1 using red ginseng is provided. More particularly, the present invention provides a method for inducing defective nef genes in HIV-1, which comprises administering an effective amount of red ginseng to a subject in need thereof. This method can derive gross deletion from the nef genes in HIV-1 and can thus be effectively applied in the prevention and treatment of AIDS.

### BACKGROUND OF THE INVENTION

1. Field of the Invention

The present invention relates to a method for inducing defective nef genes in HIV-1 using red ginseng.

2. Description of the Related Art

Human Immunodeficiency VirusType 1 (HIV-1) is a pathogen causing Acquired Immunodeficiency Syndrome (AIDS). HIV-1, along with HIV-2, belongs to a member of the lentiviridae family of retroviruses, and an infectious virion has a RNA(+) genome with two identical single-strands. When a cell is infected with HIV-1, RNA is transformed into double-stranded DNA by a reverse transcriptase coded by the RNA(+) genome, and this DNA is inserted into a chromosome of a host cell to form a provirus.

HIV-1 genome (9.2 kb) has structural genes such as gag, pol, env, and accessory genes such as tat, rev, nef, vif, vpu, vpr.

Among these genes, the nef gene is known to make a protein of about 27 kDa with its N-terminal myristoylated. The function of the nef gene is not yet known exactly, but there is considerable situational evidence that the nef protein plays a very important role in the pathogenesis of AIDS and virus growth.

First, Kestler et al. reported that, in the case of the simian immunodeficiency virus (SIV), the nef gene causes AIDS and plays an important role in virus growth (Kestler et al., Cell, 65:651, 1991). It was found that, when the simians were infected with wide-type nef (hereinafter, referred to as “nef+”) and mutants nef (point or deletion mutation in the wild type nef), only the nef+ mutants caused AIDS in the simian while the nef-deleted mutants did not cause disease at all. Meanwhile, it was found that other nef-point mutants caused AIDS with a low probability, but all of these viruses were transformed into nef+ due to reverse mutation on nef when they were recollected from the simian. This means that, in the case of SIV causing AIDS in the simian, nef plays a very important role in virus growth and the pathogenesis of AIDS, and thus the conventional hypothesis that nef is a negative factor is false.

Second, according to serological investigations, it is known that most patients and asymptomatic carriers have a large number of antibodies against the nef protein, and a large number of proteins are expressed from an initial phase of infection of AIDS patients and HIV carriers. Accordingly, it can be seen that the viruses actively express nef genes in vivo.

Third, HIV-1, HIV-2, SIV, and so forth have complete open reading frames of nef genes. The nef gene has a nucleotide of 600 to 800 bp depending on its kind, and the presence of the long open reading frame without a stop codon indirectly proves the importance of the gene.

Fourth, according to several recent reports, the nef protein is known to down-regulate a CD4 cell surface protein. The mechanism is not yet clear, but this can be considered as important evidence of an association between the nef and the pathology of AIDS.

The above evidence shows that the nef gene plays an important role in pathogenesis of AIDS and virus growth by means of a certain mechanism that has not yet been ascertained.

Panax ginseng C. A. Meyer (Panax schinseng Nees) is a perennial plant belonging to a ginseng genus of Araliaceae, and has been used in East Asian countries for several thousands of years to treat various diseases. Ginseng is known through many pharmacological experiments to have activity in relation to cholesterol reduction, lipid peroxidation suppression, blood pressure lowering, blood flow increasing, cerebrovascular expansion, cardiac hyperfunction, antiarrhythmia, antithrombus, chronic renal failure treatment, immunoregulatory function, memory enhancement, cerebral hyperfunction, antistress, antioxidation, antiaging, antiulcer, gastric juice secretion suppression, antidiabeties, detoxication, hepatocellular enzyme increase, asthma treatment, antiinflammation, analgesic function, anemia treatment, fertility enhancement, decrease of blood alcohol concentration, antiallergy, and antitumor activity.

Red ginseng is produced by heating raw ginseng with steam and then drying it to contain no more than 14% moisture. The red ginseng maintains its original hard shape and has a dark brown color due to a browning reaction during production. Through processing and dehydration, the red ginseng is protected from contamination by bacteria, fungus, and microorganisms, and rendered easier to store and transport due to its reduced volume and weight. Quality management and the identification of saponin as an important active ingredient of ginseng have led to maximizing the decomposition of saponin and additionally generating active ingredients such as maltol and ginsenoside Rh2. To date, a considerable amount of research has been conducted, both domestically and internationally, into active ingredients of red ginseng and their pharmacological effects. In particular, it has been reported that red ginseng has antioxidation activity (see Korean Society of Ginseng by Young Hun KWON, 24(1), 29-34, 2000).

### SUMMARY OF THE INVENTION

The present inventors conducted studies on a method of treating AIDS, and as a result that the nef genes in HIV-1 are defective when the red ginseng is administered over a long period to patients infected with HIV-1, thereby completing the present invention.

Therefore, it is an object of the present invention to provide a method for inducing defective nef gene in HIV-1, which comprises administering an effective amount of red ginseng to a subject in need thereof.

To achieve the above object, the present invention provides a method for inducing defective nef gene in HIV-1, which comprises administering an effective amount of red ginseng to a subject in need thereof.

Hereinafter, the present invention will be described in detail.

### BRIEF DESCRIPTION OF THE DRAWINGS

The foregoing and other objects, features and advantages of the invention will become apparent from the following more particular description of preferred embodiments of the invention, as illustrated in the accompanying drawings. The drawings are not necessarily to scale, emphasis instead being placed upon illustrating the principles of the invention.

FIG. 1 shows the clinical characteristics of 10 Long-Term Slow Progressors (LTSP) infected with HIV-1 subtype B.

FIG shows the frequency of grossly deleted nef gene (gΔnef) in the 10 LTSP.

FIG. 3 is a graph showing the changes in CD4 T cell counts in the 10 LTSP without antiretroviral therapy. A bar indicates intake periods of Korean Red Ginseng (KRG). The samples used for the sequencing of nef gene and revealing gΔnef are marked with upward (↑) and downward arrows (↓) at the bottom, respectively.

FIGS. 4A to 4C show the characteristics of gΔnef in 10 LTSP.

FIGS. 5A to 5D show the predicted amino acid sequences of representative nef proteins of 10 Korean LTSP with HIV-1 infection. The consensus amino acid sequence for Nef proteins derived from nonprogressors in the USA is shown in the top line (from Ref. 23) and the amino acid sequences predicted for 10 LTSP are aligned below. Italic letters indicate variable regions, dots indicate sequence identity, bars indicate gaps or gross deletions outside the variable region, and asterisks indicate premature stop codons.

FIG. 6 is a graph showing comparing the frequency of gΔnef of LTSP with that of a control group according to KRG intake (A), and a graph showing an occurrence rate of gΔnef according to the duration of KRG intake.

### DETAILED DESCRIPTION OF THE INVENTION

The present invention is characterized in that intake of red ginseng induce grossly deleted nef gene in HIV-1 closely associated with the pathogenesis of AIDS and virus growth.

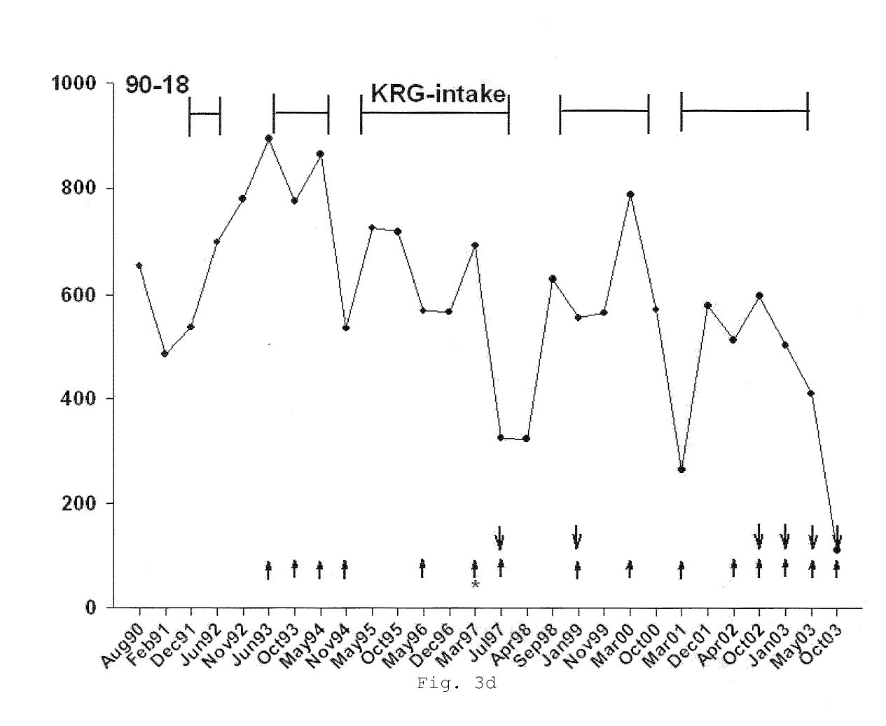

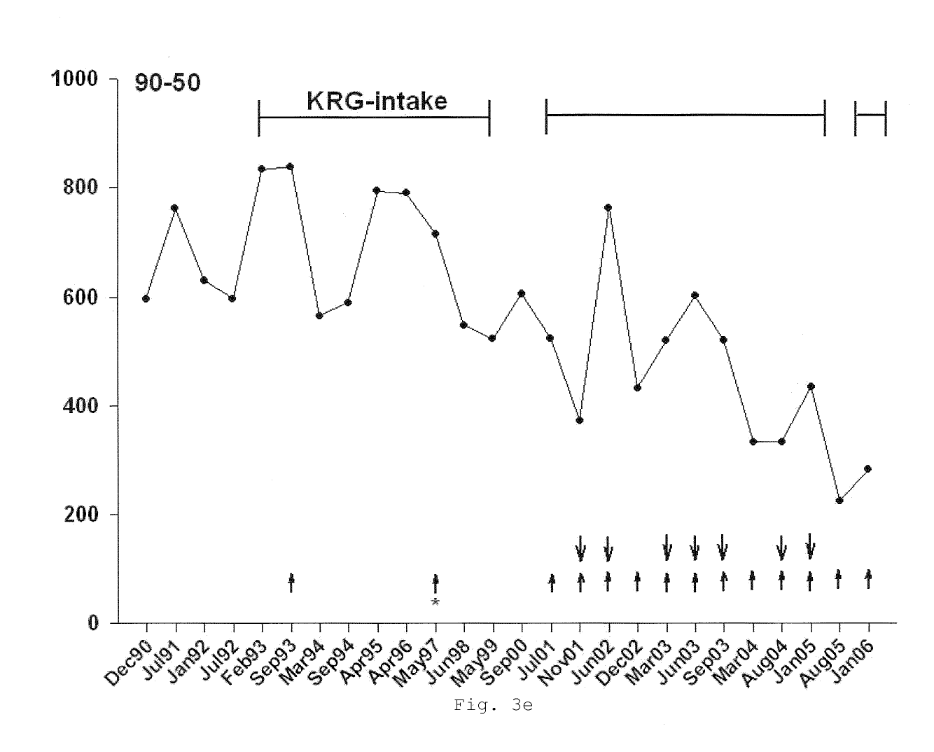

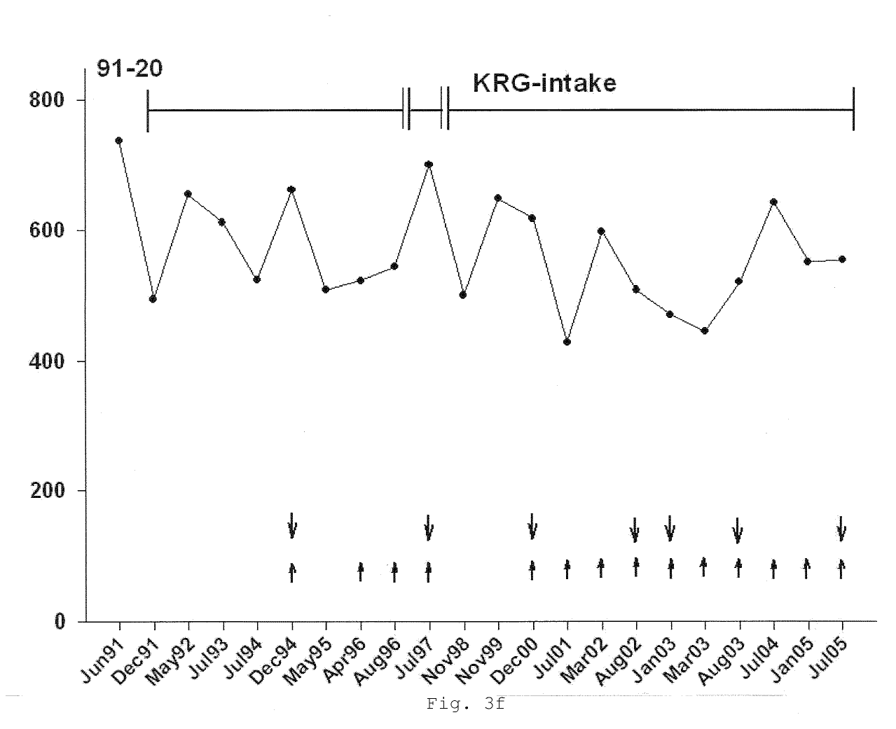

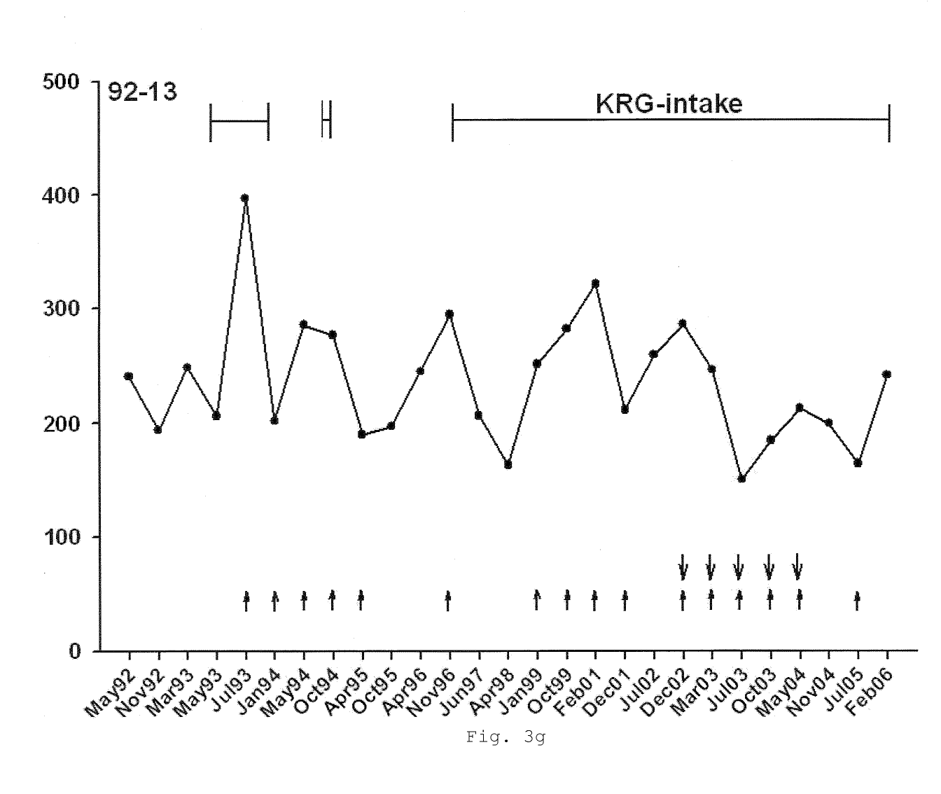

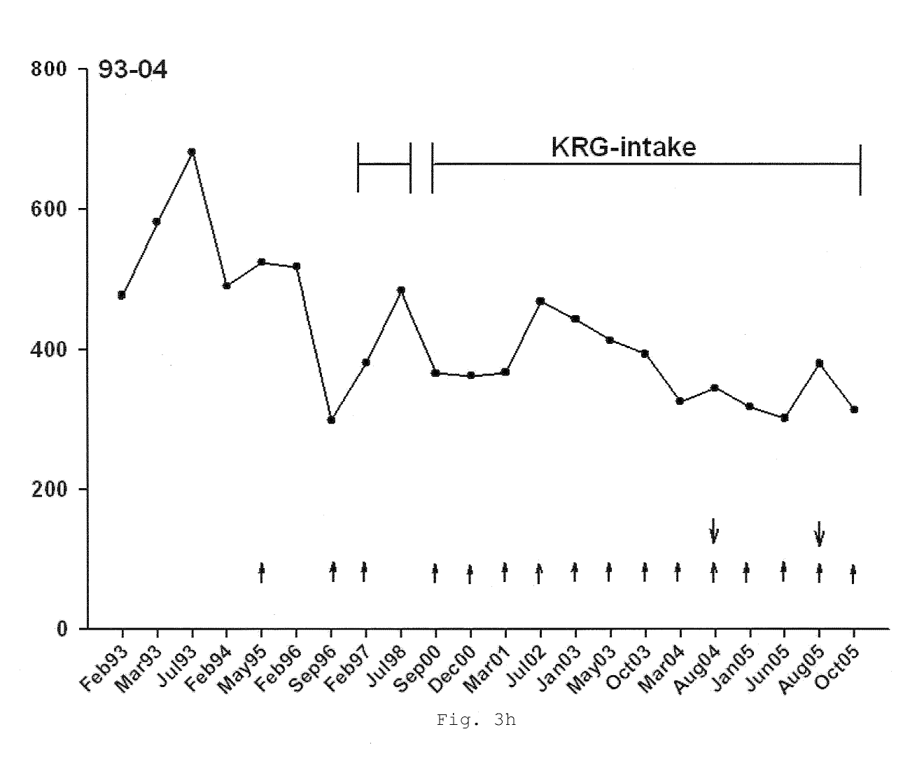

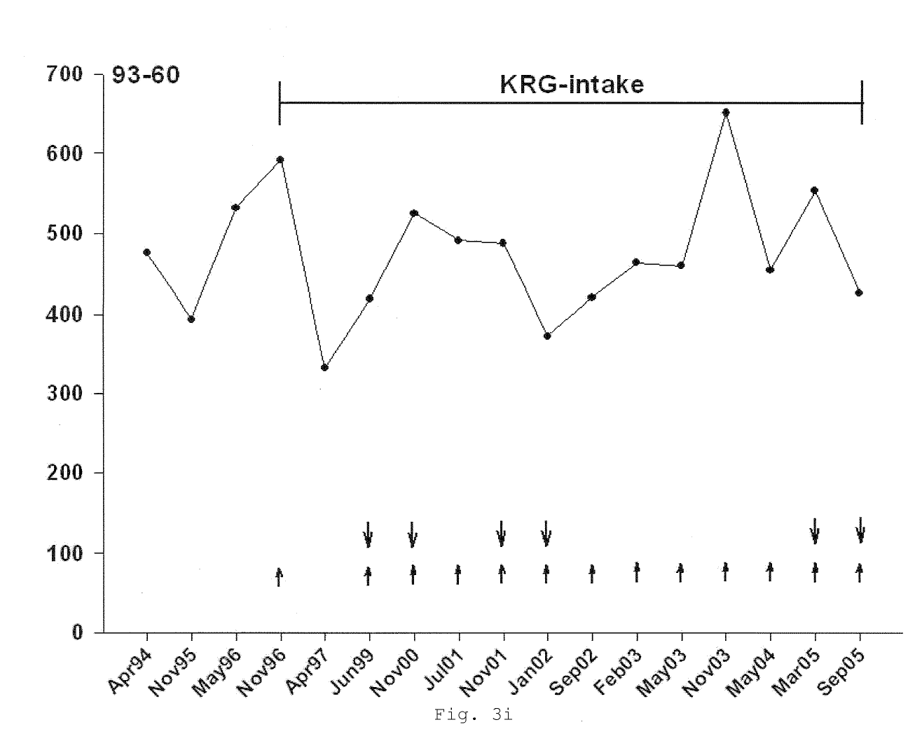

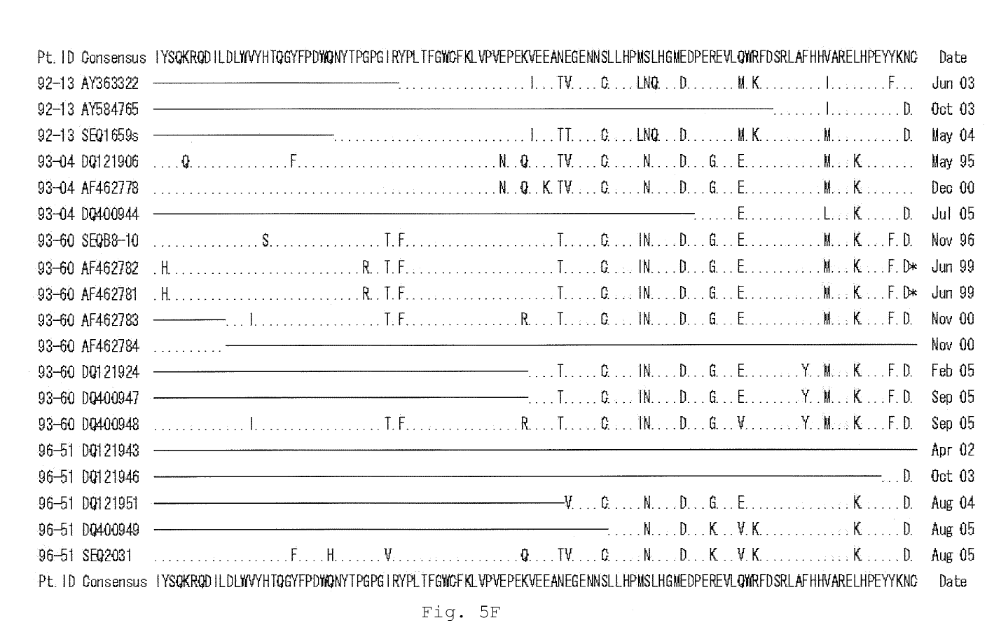

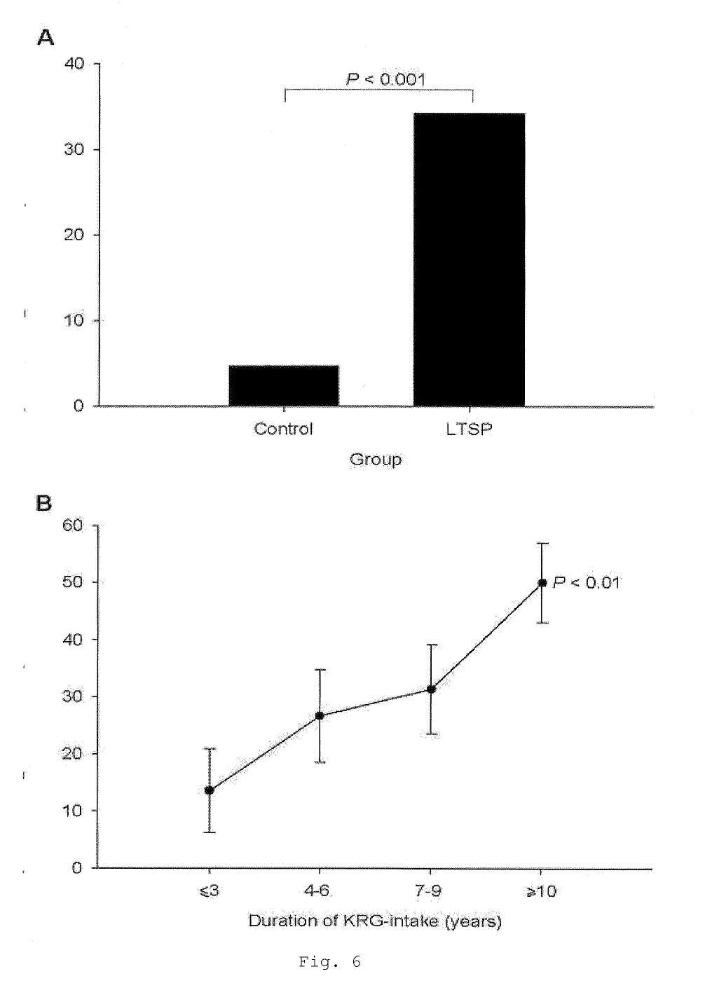

In one embodiment of the present invention, an occurrence rate of nef gene deletion (gΔnef) according to intake of red ginseng was investigated (see Example 3). As a result, nine of the 10 patients and 47 (34.3%) out of the 137 samples revealed gΔnef at many time points (see FIG. 2). In addition, of the 479 nef gene-amplified products, 90 (18.8%) were gΔnef (see FIGS. 4A to 4C). On the other hand, 4 gΔnef (2.6%) were obtained from 2 samples (4.8%) after 154 nef genes were amplified from 42 samples obtained from 34 patients of a control group without subjecting to red ginseng intake and antiretroviral therapy. Accordingly, it can be seen that the proportion with gΔnef is significantly higher in 10 patients (34.3%) than in the control group (4.8%) in the all levels of patient, sample, and gene (P<0.001, see A of FIG 6).

Therefore, grossly deletion of the nef genes in HIV-1 can be induced by administering the red ginseng to a subject, and such a method of the present invention can be effectively applied for prevention and treatment of AIDS.

The red ginseng used in the present invention can be produced by any well-known method.

In general, red ginseng is produced by a step of heating raw ginseng (containing about 75% moisture) with steam at a constant temperature, and a step of drying the heated ginseng. The resultant red ginseng can be heated and extracted to produce red ginseng extract, or the extract can be concentrated to produce a red ginseng concentrated liquid.

Meanwhile, the red ginseng extract or the red ginseng concentrated liquid may be used alone, or at least one of a carrier, an excipient, and a diluent, which are pharmacologically acceptable compositions, may be added thereto and then formulated. The term “pharmacologically acceptable” as used herein means a composition that can be physiologically acceptable and does not generally cause allergic reaction such as gastrointestinal disturbance and dizziness or a similar reaction when administered to a human being. In addition, the above-formulated red ginseng of the present invention may be administered by an appropriate method. Such a method may be oral administration.

Preferably, the red ginseng may be formulated by a method well known in the art for oral administration. For example, the red ginseng may be prepared as powder, granules, tablets, pills, sugar-coated tablets, capsules, liquid, gel, slurry, or suspension. Also, an active ingredient may be combined with a solid excipient, pulverized, augmented with an appropriate juvantia, and processed as a granular compound to obtain tablets or sugar-coated tablets. Appropriate examples of an excipient may include the saccharide such as lactose, dextrose, sucrose, sorbitol, mannnitol, xylitol, erythritol and maltitol, the starch such as corn starch, wheat starch, rice starch and potato starch, the cellulose such as cellulose, methyl cellulose, natrium carboxymethylcellulose and hydroxypropylmethyl-cellulose, and filler such as gelatin and polyvinylpyroolidone. In addition, crosslinking polyvinylpyrrolidone, agar, alginic acid, or natrium alginate may be added as a disintegrant if necessary. Further, the red ginseng of the present invention may be further including an anticoagulant, a lubricant, a humectant, an odoriferous substance, an emulsifier, or an antiseptic.

In addition, the red ginseng of the present invention may be commercially available. A red ginseng powder capsule which can be purchased from the Korea Ginseng Corporation was used in an embodiment of the present invention.

The term “subject” as used herein means a mammal and particularly an animal including a human being. Preferably, it means a patient infected with HIV-1.

In addition, the term “effective amount” as used herein means an effective amount for inducing defective nef genes in HIV-1 in vitro or in vivo.

The effective amount of red ginseng of the present invention may be determined in accordance with the above specific uses in consideration of various factors such as administration route, number of treatments, age, weight, health state, gender, severity of disorder, and food consumption and excretion rate of the subject in need of treatment. Preferably, the effective amount of the red ginseng is 60 to 90 mg/kg body weight/day.

Administration of the red ginseng may be prolonged to delay the progression to AIDS after diagnosing infection in the subject in accordance with the present invention. It may be administered for a long period, preferably, 5 years or more, and more preferably, 10 years or more.

The term “prevention” as used herein means that the danger of AIDS is reduced or suppressed. The term “treatment” as used herein means improvement, alleviation, or complete recovery from symptoms.

As a result of evaluation of the degree of deletion in nef genes according to the duration of KRG intake (see Example 4), the occurrences of gΔnef was 13.6%, 26.7%, 31.4%, and 50% for durations of ≦3, 4-6, 7-9, and ≧10 years, respectively (P<0.01; see B of FIG. 6). Accordingly, it was found that deletion in nef genes increased as the duration of KRG intake prolongs.

Meanwhile, in the present invention, a defective nef gene can be defined as one with a premature stop codon, one lacking an initiation codon, or one with 15 or more nucleotides deleted outside the variable region.

Characteristics of gΔnef derived from 90 gene-amplified products generated from 9 patients were examined in embodiments of the present invention (see Examples 5 and 6). As a result, sizes of deletions in each patient and in 9 patients were all different (see FIG. 4). To detail this, of 81 nef gene products sequenced, 56 revealed deletion within nef gene only, and in 25 from 6 patients revealed deletion extended outside the nef gene. In addition to gross deletions in the nef gene, genetic defects such as non-methionine initiation codons, premature stop codons and small deletions were observed from the amplified products of patients who took the red ginseng.

Preferably, defective nef genes by the method of the present invention may be represented as the following sequence:

GenBank accession no. AY363362, DQ121683, DQ121685, DQ121686, DQ121718, AY260799, DQ121723, DQ121724, DQ400934, DQ400937, AY221661, AY260802, AY260801, DQ121742, DQ339428, DQ121744, DQ400939, DQ121778, DQ121780, DQ121781, DQ121782, DQ121786, DQ121788, DQ121828, DQ121831, DQ121832, DQ339434, DQ121840, DQ400942, AY221690-91, AY363319, AY363321, AY363322, AY584765, DQ400944, AF462781, AF462783, AF462784, DQ121924, DQ400947, DQ121943, DQ121946, DQ121951, DQ400949.

The present invention will now be described more fully hereinafter with reference to the accompanying drawings, in which exemplary embodiments of the invention are shown. This invention may, however, be embodied in different forms and should not be construed as limited to the embodiments set forth herein. Rather, these embodiments are provided so that this disclosure will be thorough and complete, and will fully enable those skilled in the art to embody and practice the invention.

### EXAMPLE 1

### Patients and KRG Treatment

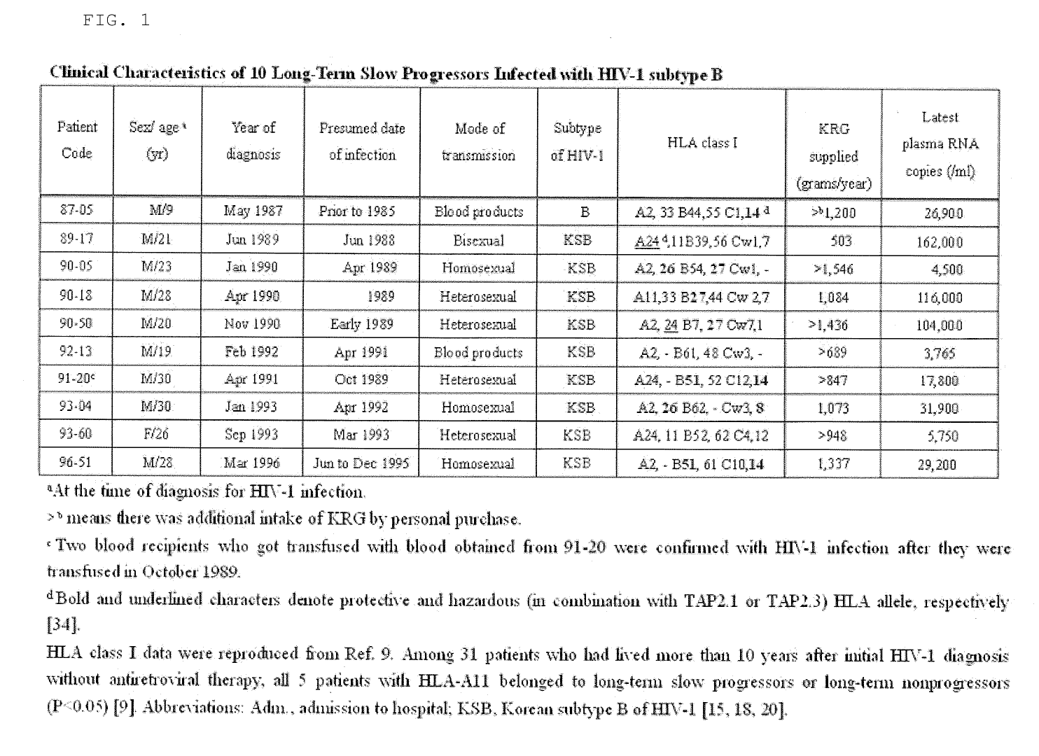

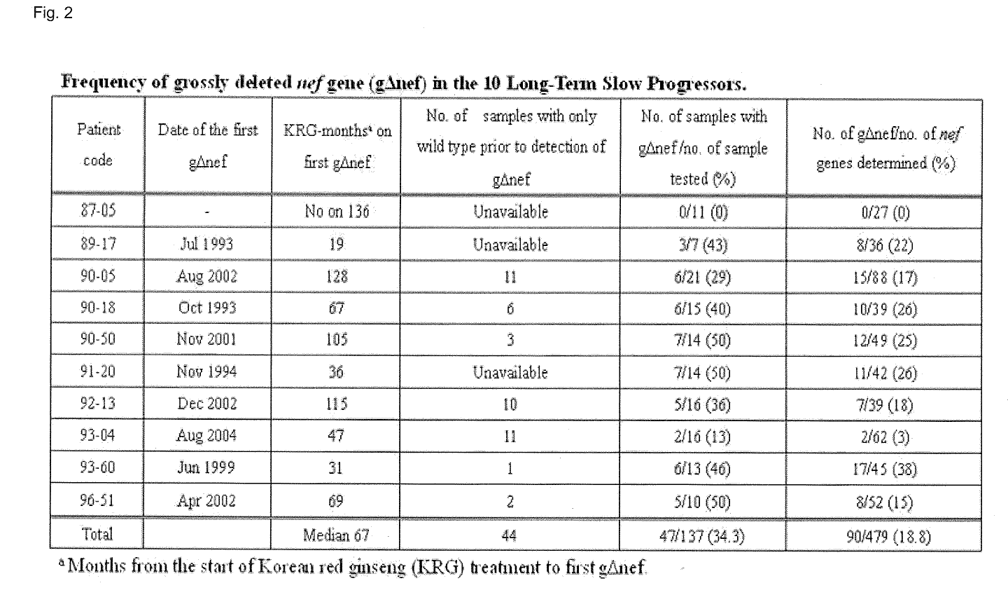

The present inventors defined a Long-Term Slow Progressor (LTSP) as a patient whose annual decrease in CD4 T cells was less than 20/μl over 10 years in the absence of antiretroviral therapy. According to these criteria, 14 patients were classified as LTSP. Of these 14 patients, 11 were infected with subtype B. One of the 11 patients was excluded due to limited sample availability for nef gene determination. Nine LTSPs were diagnosed between 1987 and 1993, and one was diagnosed in March 1996. FIGS. 1 and 2 present the details of the 10 LTSPs. There was no antiretroviral therapy in the present study period except for patient 93-04, who was intermittently treated with ZDV (daily dose of 200 mg or 300 mg) from April 1993 to August 1997. Thirty-four patients without KRG intake and antiretroviral therapy formed a control group. Informed written consent was obtained from each participant.

The KRG treatment in HIV-1-infected patients was initiated at the Korean NIH in late 1991. A red ginseng powder capsule which can be purchased from the Korean Ginseng Corporation was used for testing the KRG For each patient, the daily dose of KRG was 5.4 g for male patients and 1.8 g a day for female patients. Male patients took six 300 mg capsules three times a day. The average amount of KRG supplied to the 10 LTSPs was 11,629±5,040 g for 135.6±23 months (see FIG. 2).

### EXAMPLE 2

### Population Study

CD4 T cell counts were measured about every 6 months in 10 LTSPs of the Example 1 by the following method. Blood was drawn from each patient and peripheral blood mononuclear cells (PBMC) in each sample were incubated with phycoerythrin (PE)- and fluorescein isothiocyanate (FITC)-conjugated antibodies against CD4 antigen, respectively (Simultest reagent; Becton Dickinson, San Jose, Calif., USA). Levels of CD4 cells were measured by FACScan (Becton-Dickinson) flow cytometry.

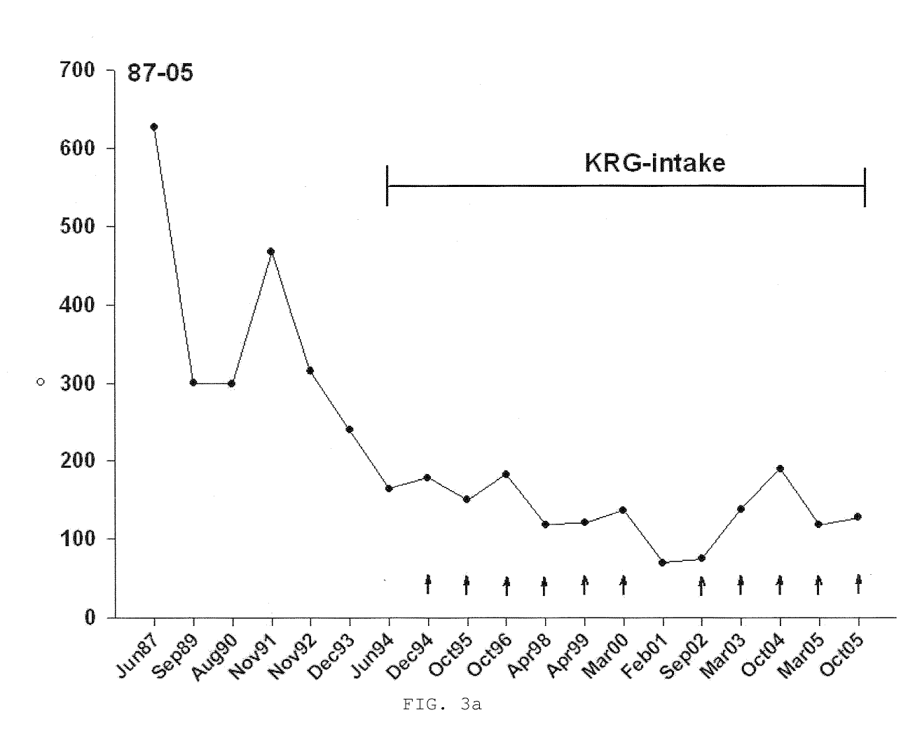

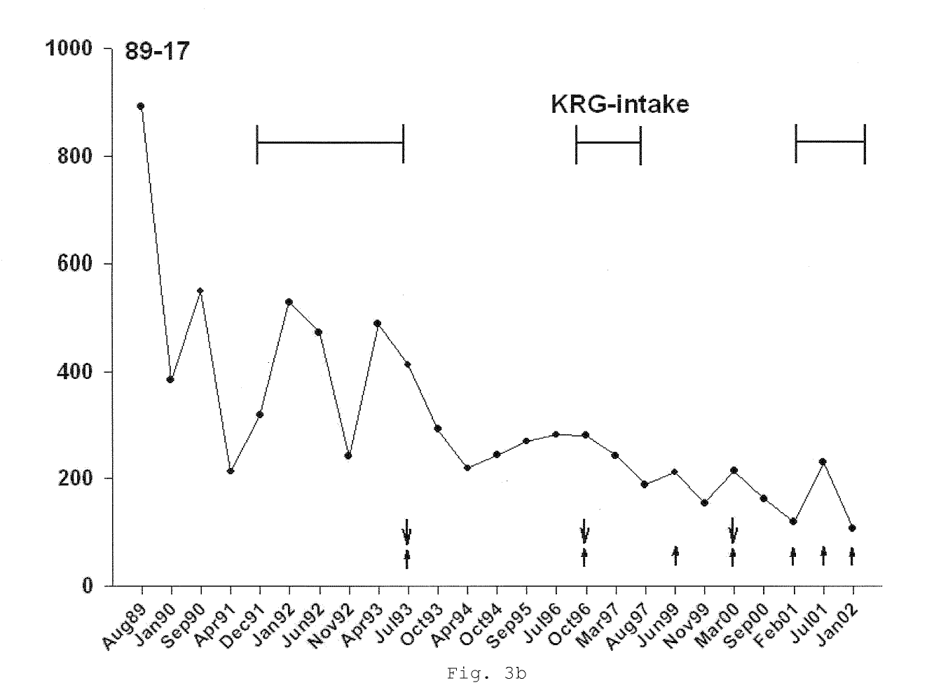

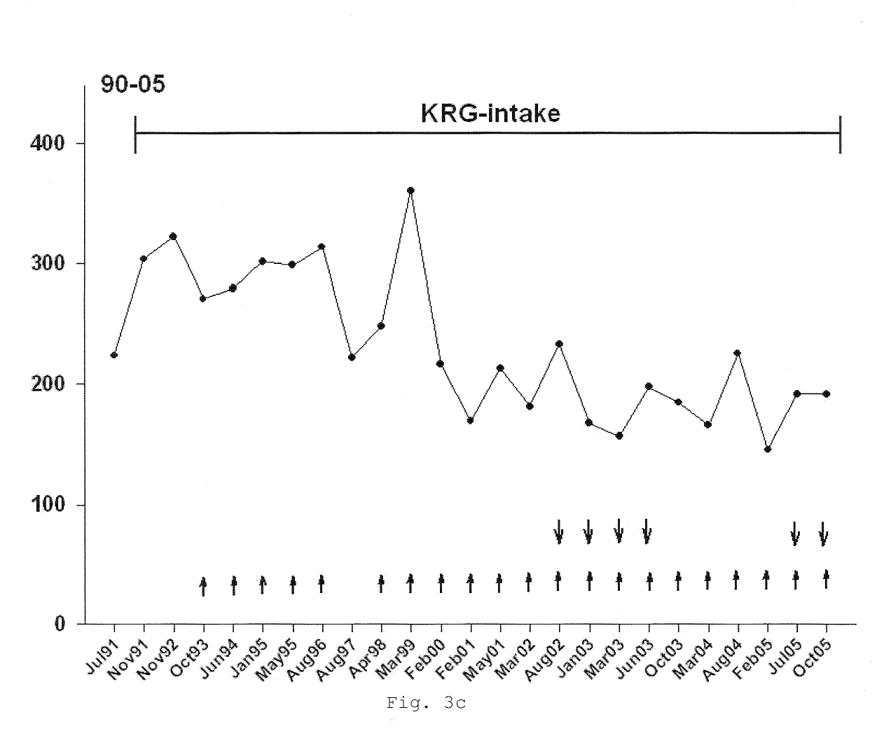

The patients were not treated with HAART, and the CD4 T cell counts decreased from 444±207 to 294±177/μl over 135.6±23 months of KRG intake. This corresponds to an annual decrease in the level of CD4 T cells of 13.3/μl with KRG intake (see FIGS. 1 and 3).

The 9 patients in this study were infected with Korean subclade B (KSB) HIV-1, and only one patient 87-05 was infected with HIV-1 derived from contaminated blood clotting factor 9 manufactured abroad. The HLA prognostic score (Sung H, et al., (2005). Clinical and Diagnostic Laboratory Immunology. 12:497-501) was −1 for patients 89-17, 0 for patients 90-50, 92-13, and 93-60, 1 for patients 87-05 and 90-18, and 2 for patients 90-05, 91-20, 93-04, and 96-51. The mean HLA prognostic score was 0.90±1.10. Although this value was not significantly higher than the value of 0.29±1.19 in 68 HIV-1 infected Korean patients (P>0.05), 4 patients (90-05, 91-20, 93-04, and 96-51) with the highest HLA score have shown better prognosis with respect to CD4 T cell counts.

### EXAMPLE 3

### Occurrence of gΔnef in LTSP According to Red Ginseng Intake

Proviral DNA extracted from uncultured PBMCs, obtained at 6-month intervals, and the nef gene in each sample, were amplified by double- or triple-nested PCR, as previously described, using the first round primers Nef5′5′ and LTR3′, the second round primers Nef5′ and N10 (Deacon N J et al., (1995) Science. 270:988-991; Rhodes D I et al., (2000) Journal of Virology, 74:10581-10588), and the third round primers Nef3 and Nef4 (Kang M R et al., (1998) Journal of Acquired Immune Deficiency Syndromes, 17:58-68; Kim Y B et al., (2003) AIDS Research and Human Retroviruses, 19:619-623) in accordance with a well-known method. A maximum of 5 PCR reactions, including a negative control group, were performed per sample at any one time. The PCR products were purified and then sequenced directly. PCR contamination was monitored by physical separation of PCR products following each procedure, such as BLAST search, comparison of amino acid codons by manual alignment, and phylogenetic analysis.

Test data were expressed as mean±standard deviation. Statistical significance was estimated by Student's 2-tailed t-test and logistic regression controlling for the subject effect (SPSS package version 12.0).

As a result, 479 nef genes were amplified from 137 PBMC samples taken from the 10 patients at different time points. Nine of the 10 patients and 47 (34.3%) out of the 137 samples revealed gΔnef at many time points (see FIG. 2). Of the 479 nef gene products, 90 (18.8%) were gΔnef (see FIGS. 4A to 4C).

Of the 9 patients with gΔnef, 7 only the wild-type (WT) product was revealed prior to detection of gΔnef. In these 7 patients, a total of 136 WT products were obtained from a total of 44 PBMC samples prior to detection of gΔnef (see FIG. 2). Samples were unavailable for the remaining 2 patients (89-17 and 91-20) prior to detection of gΔnef. The median time from commencement of KRG intake to the first detection of gΔnef was 67 months (range; 19-131) (see FIG. 2). In particular, the WT nef gene appeared consistently in 4 patients (90-05, 90-18, 92-13, and 93-04) before the detection of gΔnef, suggesting that there is an association between gΔnef and KRG intake. Detailed occurrence rates of gΔnef in each patient are as follows.

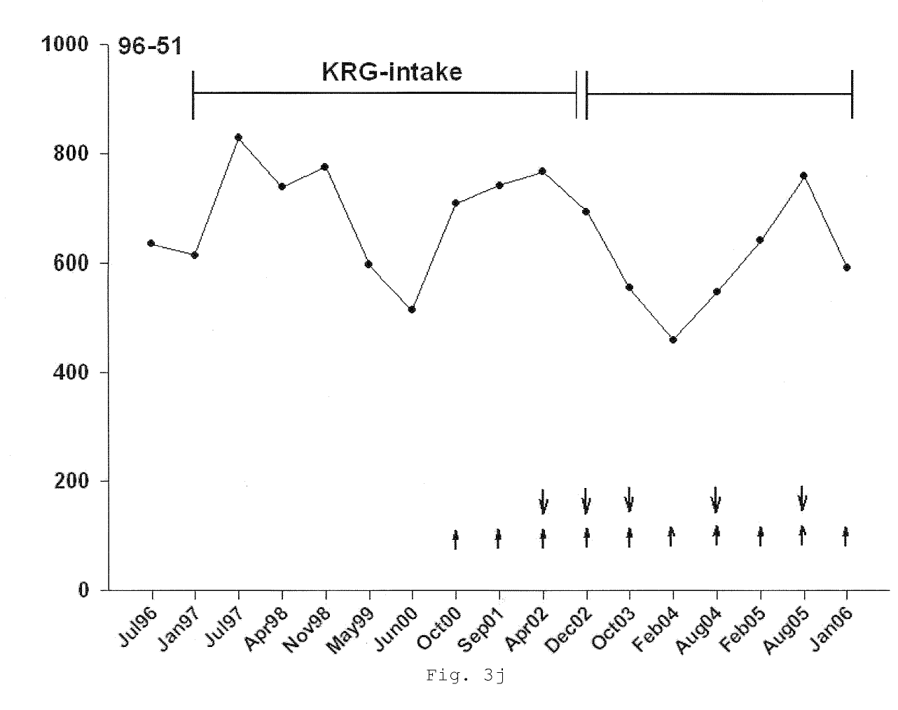

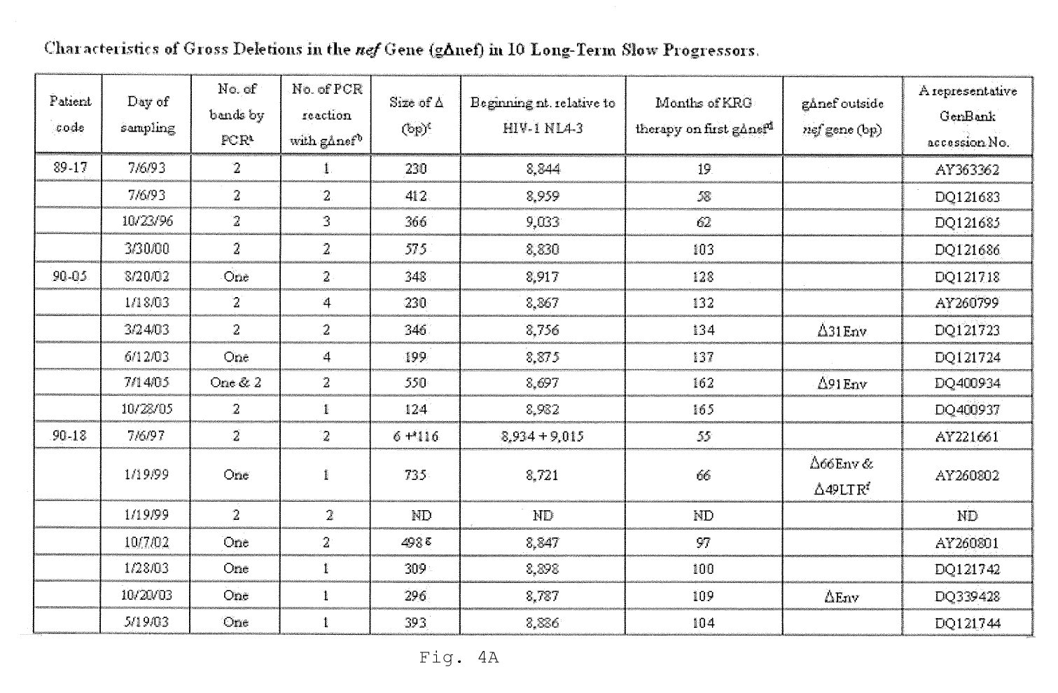

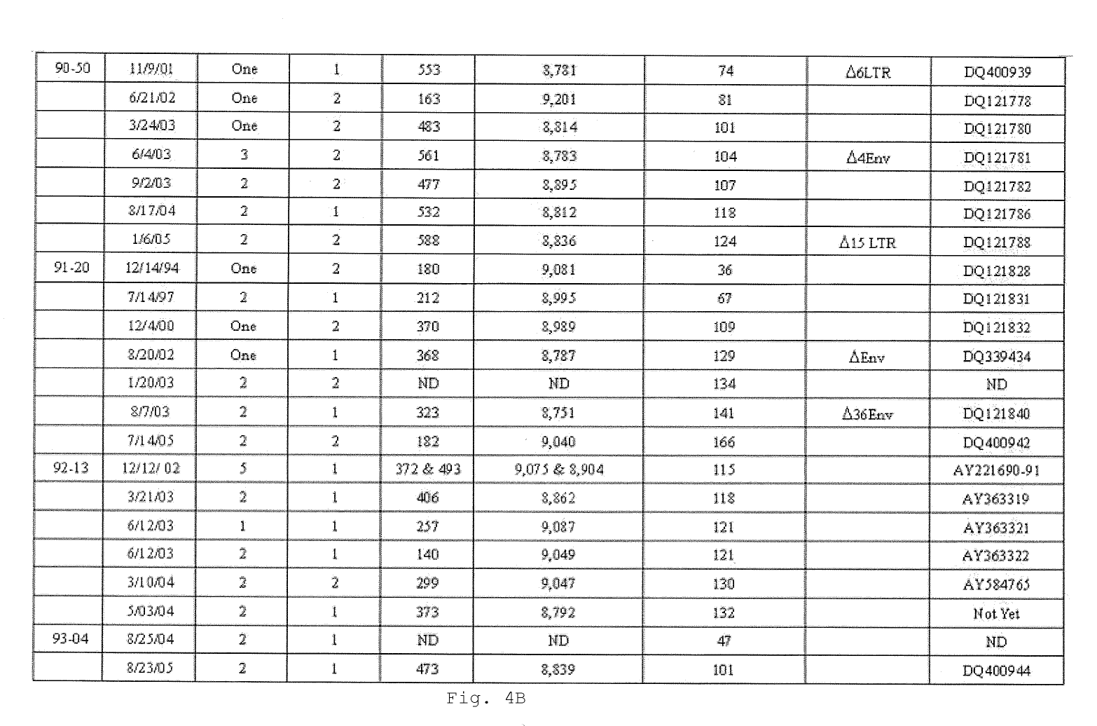

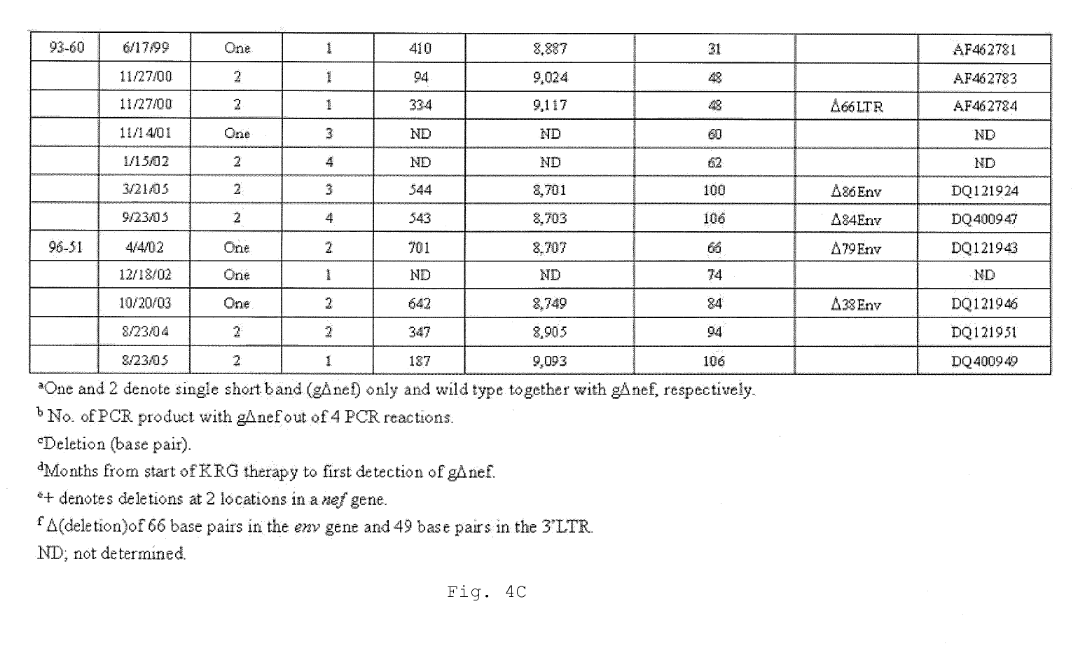

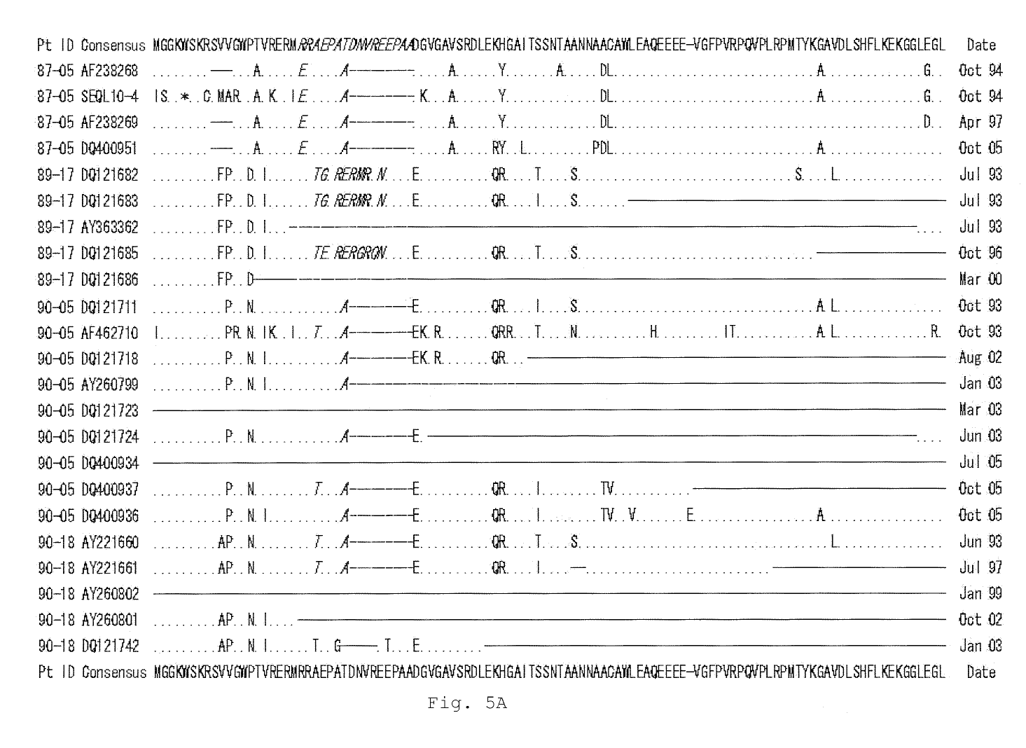

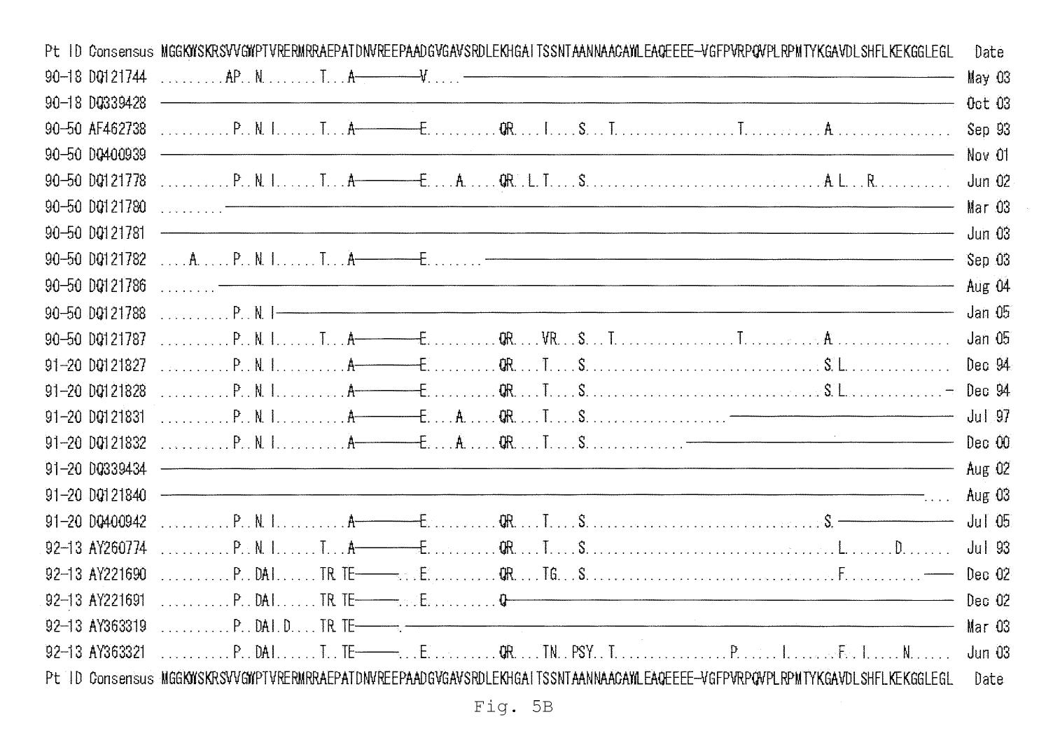

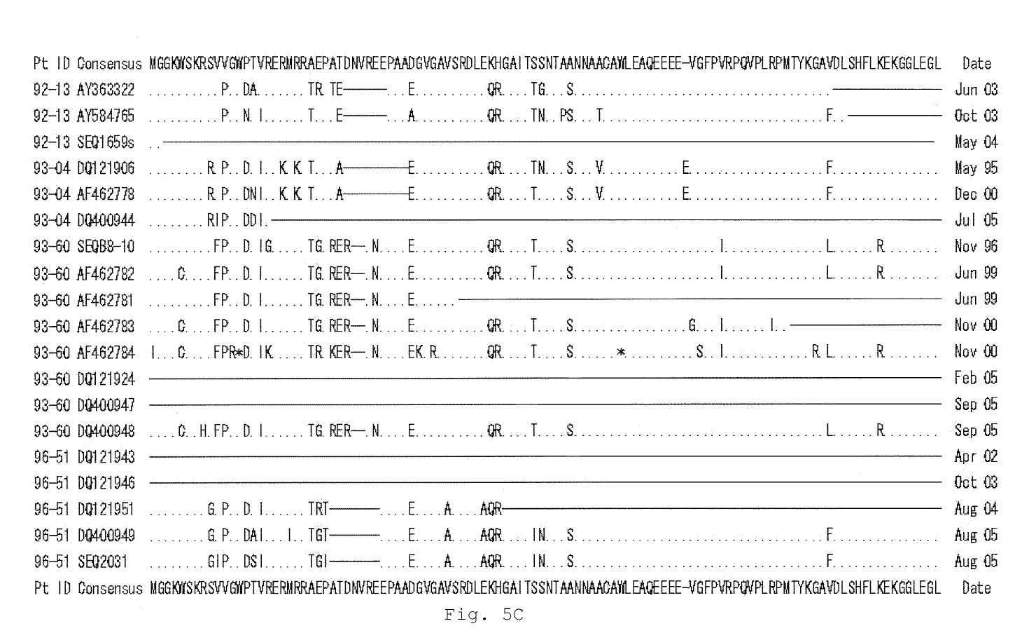

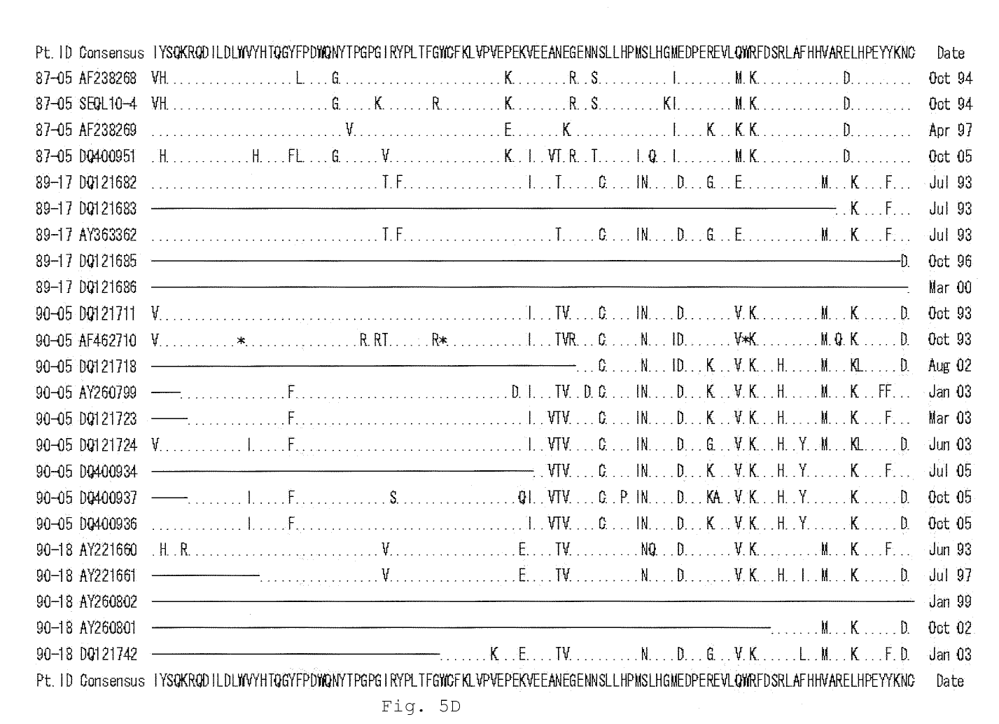

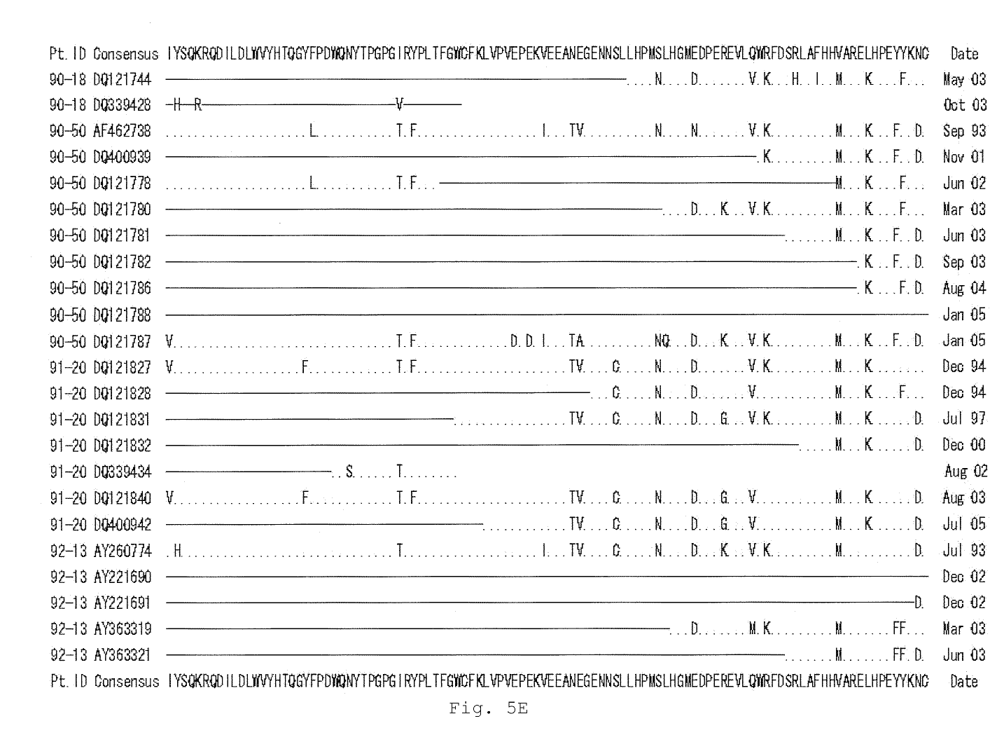

Patient 87-05 was co-infected with HCV and HIV-1 subtype B derived from contaminated clotting factor 9. The patient began treatment with KRG in June 1994, and compliance was good. Although CD4 T cell counts were low during KRG intake, the counts have been well maintained for 11 years compared with changes in the previous 7 years without KRG intake. To date, the patient has not revealed any AIDS-related symptoms. A 9 bp deletion was detected at the same location in 25 out of 27 nef genes, and a premature stop codon was detected (see FIGS. 5A to 5D).

Patient 89-17 began treatment with KRG in December 1991, and compliance was not consistent. The present inventors amplified 36 nef genes over 7 time points of from July 1993 to January 2002, and 8 gross deletions in nef genes were detected.

Patient 90-05 was diagnosed with HIV-1 infection in February 1990 and began treatment with KRG in December 1991. This patient showed the best complicance for KRG therapy in the present invention. In addition to the 21,522 g of KRG supplied, the patient self-administered KRG from personal purchases. The first instance of gΔnef was detected in August 2002, and 14,982 g of KRG was supplied for 128 months from initiation of KRG treatment. Samples taken prior to August 2002, when gΔnef was first detected, were repeatedly checked for the presence of gΔnef but there was no sign of gΔnef (see FIG. 1).

Patient 90-18 was diagnosed with HIV-1 infection in May 1990 and began treatment with KRG in December 1991, however compliance with treatment was not consistent. The first instance of gΔnef was detected in July 1997, 67 months from initiation of KRG treatment supplying 7,212 g of KRG (see FIG. 2). The CD4 T cell count for this LTNP patient maintained over 500/μl up until January 2003, decreased to 408/μl in May 2003, and then decreased to 112/μl in October 2003. Plasma HIV-1 RNA copies were maintained at less than 15,000/ml until the year 2000 and then increased to 267,000/ml and 115,000/ml in December 2001 and January 2003, respectively.

Patient 90-50 was diagnosed with an HIV-1 infection in December 1990 and began treatment with KRG in February 1993. Samples obtained before September 1993 were unavailable (see FIG. 2). WT nef genes were detected in the absence of gΔnef in July 2001 (6 nef genes). The patient did not take KRG for about 2 years prior to July 2001. The first instance of gΔnef (1 out of 5 genes) was detected in 6 consecutive samples in November 2001 (see FIG. 2). CD4 T cell counts were 434/μl (26.3%) in January 2005 (total KRG, 17,256 g).

Patient 91-20 was diagnosed with an HIV-1 infection in June 1991 and began treatment with KRG in December 1991, and compliance with treatment was consistent. The patient donated whole blood on Oct. 28, 1989 (Cho Y K et al., (1996) Journal of Korean Society of Microbiology. 31:353-360). The present inventors first detected gΔnef on Dec. 14, 1994 after 36 months of KRG treatment supplying 1,812 g of KRG (see FIG. 4). CD4 T cell counts were maintained over 553/μl (28.8%) in July 2005 (total KRG supplied, 17,800 g), and viral loads were 5,825 to 17,800 RNA copies/ml of plasma from July 1997 to March 2003.

Patient 92-13 began treatment with KRG in May 1993. Anti-HIV-1 antibody was negative in April 1991. HIV-1 infection was confirmed in February 1992 after injection of domestic blood clotting factor 9. The first CD4 T cell count was 241/μl (13.1%) in May 1992 and a baseline CD4 T cell count for KRG intake was 206/μl (16.5%). The CD4 T cell count was maintained well for 14 years. The present inventors could detect gΔnef in December 2002. The amount of KRG supplied was 8,790 g, and there was additional amount by personal purchases.

Patient 93-04 was confirmed to have an HIV-1 infection in April 1992. In February 1997, following self-administration of KRG from a personal purchase, the CD4 T cell count was 380/μl (33.9%). The present inventors started to supply the KRG from September 2000. The patient also took independently purchased KRG The present inventors obtained 30 WT nef only until March 2004. The first instance of gΔnef was detected in August 2004.

Patient 89-17, the wife of patient 93-60, was diagnosed with HIV-1 infection in October 1993. According to medical records at the time of initial infection, primary infection related symptoms appeared in March 1993. The patient began treatment with KRG in November 1996. As the only female, the patient could not tolerate the daily dose of 5.4 g which caused facial flushing, and thus took a smaller dose of 1.8 g (mainly) to 3.0 g since 1998.

Patient 96-51 was diagnosed with HIV-1 infection in April 1996. The CD4 T cell count and CD4/CD8 ratio were 634/μl and 1.13 in July 1996. The first 2 samples, taken in October 2000 and September 2001, contained 14 WT nef genes. In samples taken in April 2002, the present inventors detected gΔnef in 1 out of 5 nef genes (DQ121943)(see FIG. 4).

In addition, the present inventors amplified 154 nef genes from 42 samples obtained from the 34 control patients without KRG intake and antiretroviral therapy. Among those, the present inventors obtained 4 gΔnef (2.6%) in 2 samples (4.8%) from 2 patients (5.9%). Therefore, in terms of patient, sample, and gene, the proportion with gΔnef was significantly higher in 10 LTSPs (34.3%) than in the control group (4.8%) (P<0.001, see A of FIG. 6).

### EXAMPLE 4

### Relationship Between the Occurrence of gΔnef and Duration of KRG Intake

The occurrence of gΔnef among 479 nef genes in 137 PBMC samples of 10 patients was assessed in relation to duration of KRG intake. The duration of KRG intake was ≦3, 4-6, 7-9, and ≧10 years, and 22, 30, 35, and 50 PBMC samples in the same order were used to amplify the nef gene by the same method of Example 3. As a result, there was no detection of gΔnef in 12 samples obtained prior to 18 months of KRG-intake. The proportions of samples containing gΔnef increased according to the duration of KRG intake. The proportion was shown as 13.6%, 26.7%, 31.4%, and 50% in order of ≦3, 4-6, 7-9, and ≧10 years (P<0.01, see B of FIG. 6). In particular, gΔnef started to show after 9 years of KRG intake by 2 patients (90-05 and 92-13).

Accordingly, it was found that the occurrence of gΔnef increased in proportion to duration of KRG intake.

### EXAMPLE 5

### Nature of gΔnef

The characteristics of gΔnef from 90 products generated from 9 patients are summarized in FIG. 4. Examination of the size of the deletion relative to NL4-3 revealed various deletion types. Sequences were determined for 81 of the 92 nef gene products. Two deletion sites (6 bp+116 bp) (AY221661) in the nef gene derived from the sample obtained on Jul. 6, 1997 were revealed for patient 90-18. Apart from this case, all deletions in the nef gene were derived from a single site (see FIG. 5). Of the 81 nef gene products sequenced, 56 revealed deletion within the nef gene only and 25 revealed deletion extended outside the nef gene from 6 patients.

The deletion size relative to NL4-3 ranged from 94 bp to 735 bp (median 368 bp). Two gΔnef (AY260801 from patient 90-18 and DQ121943 from patient 96-51) revealed a deletion larger than a normal nef gene size (ranging from 621 to 630 bp) and did not contain any partial nef gene. The sizes of deletions in each patient and in 9 patients were all different (see FIG. 4).

### EXAMPLE 6

### Genetic Defects and Deletions smaller than gΔnef

In addition to gross deletions in the nef gene, the present inventors observed genetic defects such as non-methionine initiation codons, premature stop codons, and small deletions. Patient 87-05 had a 9 bp deletion in 25 out of 27 nef genes, isoleucine as an initiation codon, and a premature stop codon in another nef gene. A non-methionine initiation codon and 3 premature stop codons were detected in the nef gene in October 1993 in patient 90-05. Patient 93-60 had a non-methionine initiation codon and 2 premature stop codons in the nef gene in November 2000, a premature stop codon in July 1993, and deletion of the last cysteine in the nef gene in June 1999.

According to the present invention as described above, administering red ginseng to a subject infected with HIV-1 can derive deletion from nef genes in HIV-1. Therefore, the present invention can be effectively applied for prevention and treatment of AIDS.

While the invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method for inducing defective nef gene in HIV-1, which comprises administering an effective amount of red ginseng to a subject in need thereof.

2. The method of claim 1, wherein the subject is a patient infected with HIV-1.

3. The method of claim 1, wherein the effective amount is 60 to 90 mg/kg body weight/day.

4. The method of claim 1, wherein the red ginseng is administered for at least 5 years.

5. The method of claim 1, wherein the defective nef gene is selected from the group consisting of one with a premature stop codon, one lacking an initiation codon, and one deleting of more than 15 nucleotides outside the variable region.

6. The method of claim 1, wherein the defective nef gene is selected from the group consisting of GenBank accession No. AY363362, DQ121683, DQ121685, DQ121686, DQ121718, AY260799, DQ121723, DQ121724, DQ400934, DQ400937, AY221661, AY260802, AY260801, DQ121742, DQ339428, DQ121744, DQ400939, DQ121778, DQ121780, DQ121781, DQ121782, DQ121786, DQ121788, DQ121828, DQ121831, DQ121832, DQ339434, DQ121840, DQ400942, AY221690-91, AY363319, AY363321, AY363322, AY584765, DQ400944, AF462781, AF462783, AF462784, DQ121924, DQ400947, DQ121943, DQ121946, DQ121951, and DQ400949.
